(19) United States
(12) Patent Application Publication
(10) Pub. No.: US 2008/0268463 A1
(43) Pub. Date: Oct. 30, 2008

(54) Methods for Prognosis and Diagnosis of Chronic Prostatitis/Pelvic Pain Syndrome and Interstitial Cystitis

(75) Inventors: Jordan D. Dimitrakov, Boston, MA (US); Michael D. Smith, Brookline, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/043,403

(22) Filed: Mar. 6, 2008

Related U.S. Application Data
(60) Provisional application No. 60/905,425, filed on Mar. 7, 2007.

(51) Int. Cl. G01N 33/53; G01N 33/566

(52) U.S. Cl. 435/7.1; 436/501

(57) ABSTRACT

We have discovered that levels of specific protein biomarkers present in urine samples of subjects correlate with the presence, or absence of, chronic prostatitis/chronic pelvic pain syndrome (CP/CPPS) in men or interstitial cystitis (IC) in women. These protein biomarkers are cortitropin releasing hormone (CRH), dehydroepiandrosterone (DHEA), neuropeptide Y (NPY) and Galanin. Accordingly, the invention is directed to methods for diagnosis of CP/CPPS and IC by monitoring the levels of at least one of these proteins in urine, as well as to diagnostic kits designed for diagnosis of CP/CPPS and IC.

### CROSS-REFERENCE TO RELATED APPLICATIONS

This application claims the benefit under 119(e) of provisional application number 60/905,425 filed on Mar. 7, 2007.

### GOVERNMENT SUPPORT

This invention was made with Government support under grant RO1 DK65990 awarded by the National Institutes of Health (NIH). The Government has certain rights in the invnetion.

### FIELD OF THE INVENTION

The present invention relates generally to methods for the diagnosis of chronic prostatitis/chronic pain syndrome (CP/CPPS) and Interstitial cystitis (IC) by assessing the levels of biomarkers in urine; post-prostatic massage urine in men and urine in women.

### BACKGROUND OF THE INVENTION

Chronic prostatitis or chronic pelvic pain syndrome (CP/CPPS) is a debilitating condition characterized by pain or discomfort in the pelvic or perineal area and is a condition often associated with erectile dysfunction.

While CP/CPPS is a highly prevalent disease in men, with an estimated 35-50% of men affected by prostatitis at some time in life, definitive methods for the diagnosis and treatment of CP/CPPS have remained elusive.

Interstitial cystitis (IC) is the condition related to CP/CPPS in women. The chronic pain experienced by IC and CP/CPPS patients is similar to the pain experienced following injury, or inflammation of peripheral nerves. The stimuli that normally would never cause pain results in pain in these patients.

There is a need in the art to establish diagnostic and therapeutic markers to facilitate the diagnosis of this debilitating syndrome as well as to facilitate the development of therapeutics.

### SUMMARY OF THE INVENTION

The present invention is based on the discovery of specific biomarkers that are present in urine at higher or lower concentrations in patients that have chronic prostatitis/chronic pain pelvic syndrome (CP/CPPS) or Intersitial cyctitis (IC) as compared to subjects that have no symptoms of CP/CPPS or IC. In particular, cortitropin releasing hormone (CRH) and dehydroepiandrosterone (DHEA) were found to be present at higher concentrations in urine from end stage CP/CPPS patients that is voided after prostatic message as compared to post-prostatic massage urine of asymptomatic men. In addition, neuropeptide Y (NPY) and Galanin were found to be present at lower concentrations in post prostatic massage urine of men with end stage CP/CPPS as compared to healthy men. Accordingly, the invention is directed to methods for diagnosis of CP/CPPS and IC by monitoring the levels of at least one of these proteins in urine (post-prostatic massage urine of men and urine of women), as well as to diagnostic kits designed for diagnosis of CP/CPPS and IC.

In one embodiment, a method for facilitating the diagnosis of a subject for chronic prostatitis/chronic pain pelvic syndrome (CP/CPPS) in men or Intersitial cyctitis (IC) in women is provided. The method comprises measuring the level of at least one biomarker protein in a urine test sample where the biomarker protein is selected form the group consisting of cortitropin releasing hormone (CRH), dehydroepiandrosterone (DHEA), neuropeptide Y (NPY) and Galanin. A higher level of cortitropin releasing hormone (CRH) or dehydroepiandrosterone (DHEA) in the test sample as compared to a reference level (e.g. the level of the same biomarker protein in a control sample) is indicative of CP/CPPS in men or IC in women, a lower level of neuropeptide Y (NPY) or Galanin in the test sample as compared to a reference level (e.g. the level of the same biomarker protein in the control sample) is indicative of CP/CPPS in men or IC in women.

In one embodiment, the levels of at least 2, at least three 3, or at least 4, biomarker proteins are measured.

The term “test sample” refers to a urine sample obtained from a subject being tested for CP/CPPS (men) or IC (women). The test sample in order to test for CP/CPPS is obtained from a man after the man has been subjected to prostatic massage. The prostatic massage results in prostatic fluid being voided in the test urine sample. The test sample in order to test for IC in women is a urine sample obtained from a woman being tested for IC.

The term “control sample” refers to a urine sample obtained from a different subject that is asymptomatic for CP/CPPS or IC. When testing for CP/CPSS a “control sample” also refers to pre-prostatic message urine sample obtained from the same subject to be tested for CP/CPSS by using a post-prostatic massage urine sample.

The term “a reference level” refers to a level of biomarker protein that is present in a subject that is asymptomatic for CP/CPPS or IC, or in a pre-prostatic message urine sample obtained from a test subject. The “reference level” can be an average level of a biomarker protein, e.g. obtained form data from multiple subjects. Alternatively, a test sample can be compared directly to the level present in a control sample, e.g. when testing pre-prostatic massage versus post-prostatic massage urine for biomarkers. For purposes of comparison, the biomarker of the test sample is compared to a reference level for the same biomarker protein.

In one aspect of the invention, levels of biomarker protein present in a test biological sample are measured by contacting the test sample, or preparation thereof, with an antibody-based binding moiety that specifically binds to the biomarker protein, or to a portion thereof. The antibody-based binding moiety forms a complex with the biomarker protein that can be detected, thereby allowing the levels of the biomarker protein to be measured.

Antibody-based immunoassays are the preferred means for measuring levels of biomarker protein, e.g. by ELISA assay. However, any means known to those skilled in art can be used to assess biomarker protein levels. Biomarker protein levels can be assessed by mass spectrometry, including SELDI mass spectrometry. Enzyme assays can also be used.

In a further embodiment, the invention provides for kits that comprise means for measuring the biomarker proteins in a urine sample to facilitate diagnosis of CP/CPPS in men or IC in women.

The present invention further contemplates the assessment of levels of these biomarker proteins to monitor the therapeutic efficacy of a treatment regime designed to treat a patient having CP/CPPS or IC.

Other aspects of the invention are disclosed infra.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIG. 1 shows the amino acid sequence for cortitropin releasing hormone (CRH) (SEQ ID NO:1).

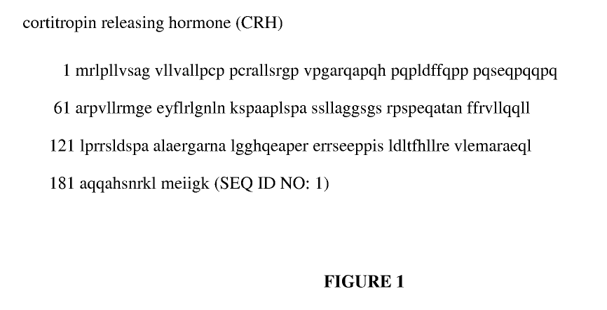

FIG. 2 shows the amino acid sequence for dehydroepiandrosterone (DHEA) (SEQ ID NO:2).

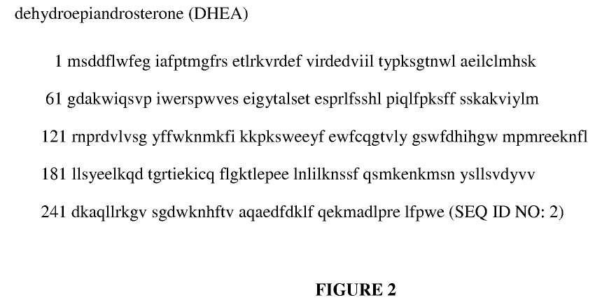

FIG. 3 shows the amino acid sequence for neuropeptide Y (NPY) (SEQ ID NO:3).

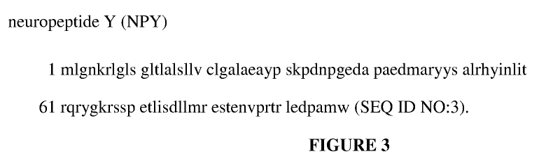

FIG. 4 shows the amino acid sequence for and Galanin (SEQ ID NO:4).

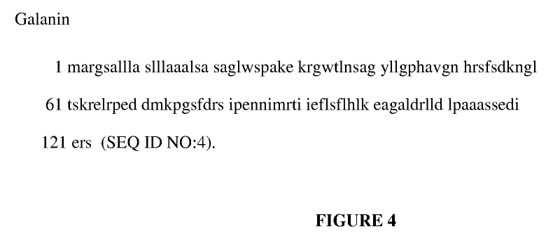

FIG. 5 shows a graph depicting levels of CRH, as measured by ELISA, in CPPS patients as compared to those in controls.

FIG. 6 shows a graph depicting levels of NPY, as measured by ELISA, in CPPS patients as compared to those in controls.

### DETAILED DESCRIPTION OF THE INVENTION

We have discovered that the levels of specific protein biomarkers present in urine samples of subjects correlate with the presence, or absence of, chronic prostatitis/chronic pelvic pain syndrome (CP/CPPS) in men or Interstitial cystitis (IC) in women. These protein biomarkers are cortitropin releasing hormone (CRH), dehydroepiandrosterone (DHEA), neuropeptide Y (NPY) and Galanin.

As used herein, “cortitropin releasing hormone” or “CRH” refers to the CRH protein of Genebank accession, protein, NP_{—}000747 (Homosapiens) (SEQ ID NO:1). The term “cortitropin releasing hormone (CRH)” also encompasses species variants, homologues, allelic forms, mutant forms, and equivalents thereof. In the methods described herein, fragments of these proteins can be detected.

“Dehydroepiandrosterone” or “DHEA” refers to the DHEA protein of Genebank accession, protein, AAA17749 (Homosapiens) (SEQ ID NO:2). The term “dehydroepiandrosterone (DHEA)” also encompasses species variants, homologues, allelic forms, mutant forms, and equivalents thereof. In the methods described herein, fragments of these proteins can be detected.

“Neuropeptide Y” or “NPY” refers to the NPY protein of Genebank accession, protein, NP_{—}000896 (Homosapiens) (SEQ ID NO:3). The term “neuropeptide Y (NPY)” also encompasses species variants, homologues, allelic forms, mutant forms, and equivalents thereof. In the methods described herein, fragments of these proteins can be detected.

“Galanin” refers to the Galanin protein of Genebank accession, protein, NP_{—}057057 (Homosapiens) (SEQ ID NO:4). The term “Galanin” also encompasses species variants, homologues, allelic forms, mutant forms, and equivalents thereof. In the methods described herein, fragments of these proteins can be detected.

The present invention is directed to methods for facilitating diagnosis of CP/CPPS in men and IC in women.

In one embodiment, the methods involve measuring levels of biomarker protein in a test sample obtained from a patient, suspected of having CP/CPPS or IC, and comparing the observed levels to levels of biomarker protein found in a control sample e.g., a sample obtained from an individual patient or population of individuals that are asymptomatic for CP/CPPS or IC. Levels of cortitropin releasing hormone (CRH) and/or dehydroepiandrosterone (DHEA) that are higher than the respective levels observed in the control indicate the presence of CP/CPPS or IC. Levels of neuropeptide Y (NPY) and/or Galanin that are lower than the respective levels observed in the control indicate the presence of CP/CPPS or IC. The levels of biomarker protein can be represented by arbitrary units, for example as units obtained from a densitometer, luminometer, an activity assay, or an ELISA plate reader.

As used herein, “a higher level of biomarker protein in the test sample as compared to the level in the control sample” refers to an amount of biomarker protein that is greater than an amount of biomarker protein present in a control sample. The term “higher level” refers to a level that is statistically significant or significantly above levels found in the control sample. Preferably, the “higher level” is at least 2 fold greater.

As used herein, “a lower level of biomarker protein in the test sample as compared to the level in the control sample” refers to an amount of biomarker protein that is lower than an amount of biomarker protein present in a control sample. The term “higher level” refers to a level that is statistically significant or significantly above levels found in the control sample. Preferably, the “higher level” is at least 2 fold greater.

As used herein, “biomarker protein” refers to a protein selected from the group consisting of cortitropin releasing hormone (CRH), dehydroepiandrosterone (DHEA), neuropeptide Y (NPY) and Galanin.

The term “statistically significant” or “significantly” refers to statistical significance and generally means a two standard deviation (2SD) above normal, or higher, concentration of the marker.

For purposes of comparison, the control sample can also be a standard sample that contains the same concentration of biomarker protein that is normally found in a post-prostatic massage urine sample that is obtained from a healthy man or in a urine sample from a healthy woman. For example, there can be a standard normal control sample for the amounts of biomarker protein.

In one aspect of the invention, a secondary diagnostic step can be performed. For example, if a level of biomarker protein is found to indicate the presence of CP/CPPS or IC, then an additional method of detecting the syndrome can be performed to confirm the presence of CP/CPPS or IC. Any of a variety of additional diagnostic steps can be used, such as direct analysis of prostatic fluid, or any other method.

### Measuring Levels of Biomarker Protein

The levels of biomarker protein can be measured by any means known to those skilled in the art. In the present invention, it is generally preferred to use antibodies, or antibody equivalents, to detect levels of biomarker protein. However, other methods for detection of biomarker expression can also be used. Biomarker protein activity, can also be measured, for example sulfotransferase activity of DHEA can be measured.

In one embodiment, levels of biomarker protein are measured by contacting the biological sample with an antibody-based binding moiety that specifically binds to the biomarker protein, or to a fragment of the biomarker protein. Formation of the antibody-biomarker protein complex is then detected as a measure of biomarker protein levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to the biomarker protein. The term “antibody-based binding moiety” is intended to include whole antibodies, e.g., of any isotype (IgG, IgA, IgM, IgE, etc), and includes fragments thereof which are also specifically reactive with biomarker protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody-based binding moiety” includes polyclonal, monoclonal, or other purified preparations of antibodies and recombinant antibodies. The term “antibody-based binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the diagnostic methods of the invention that use antibody based binding moieties for the detection of biomarker levels, the level of biomarker present in the biological samples correlate to the intensity of the signal emitted from the detectably labeled antibody.

In one preferred embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audoradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one preferred embodiment the biomarker proteins are detected by immunoassays, such as enzyme linked immunoabsorbant assay (ELISA), radioimmunoassay (RIA), Immunoradiometric assay (IRMA), or Western blotting, these assays are well known to those skilled in the art. Immunoassays such as ELISA or RIA, which can be extremely rapid, are more generally preferred. Antibody arrays or protein chips can also be employed, see for example U.S. patent application Ser. Nos: 20030013208A1; 20020155493A1; 20030017515 and U.S. Pat. Nos: 6,329,209; 6,365,418, which are herein incorporated by reference in their entirety.

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-THP Uromodulin, anti-CD13 Aminopeptidase N (AMPN), anti-CD26 Dipeptidylpeptidase IV, anti-CD10 anti-Neprilysin (NEP), Nti-ZAG ZA2G (zinc-a-2-glycoprotein) or anti-PSA ) is linked to a solid phase (i.e. a microtiter plate) and exposed to a biological sample containing antigen (e.g. biomarker protein). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing the biomarker protein (i.e. antigen). The antigen-antibody mixture is then contacted with a solid phase (e.g. a microtiter plate) that is coated with antigen. The more antigen present in the sample, the less free antibody that will be available to bind to the solid phase. A labeled (e.g., enzyme linked) secondary antibody is then added to the solid phase to determine the amount of primary antibody bound to the solid phase.

Other techniques may be used to detect the biomarkers of the invention, according to a practitioner's preference, and based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to biomarker proteins can then be used to assess biomarker levels, where the intensity of the signal from the detectable label corresponds to the amount of biomarker present. Levels can be quantitated, for example by densitometry.

### Mass Spectometry

In addition, biomarkers of the invention may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

In one preferred embodiment, biomarker levels are measured by MALDI-TOF mass spectrometry.

Detection of biomarker proteins using activity assays are also contemplated.

### Antibodies

The antibodies for use in the present invention can be obtained from a commercial source. Alternatively, antibodies can be raised against the biomarker protein to be detected, or a portion of the biomarker polypeptide. Methods useful for the production of antibodies are disclosed in U.S. application Ser. Nos. 2002/0182702; 2003/0212256; 20020110894 and WO 01/11074, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

### Detection Kits

The present invention is also directed to commercial kits for the detection and prognostic evaluation of CP/CPPS and/or IC. The kit can be in any configuration well known to those of ordinary skill in the art and is useful for performing one or more of the methods described herein for the detection of biomarker proteins. The kits are convenient in that they supply many if not all of the essential reagents for conducting an assay for the detection of biomarker protein in a urine sample, e.g. a pre and post-prostatic massage sample. In addition, the assay is preferably performed simultaneously with a standard or multiple standards that are included in the kit, such as a predetermined amount of biomarker protein, so that the results of the test can be quantitated or validated.

The kits include an assay means for detecting biomarker levels such as antibodies, or antibody fragments, which selectively bind to biomarker protein In one embodiment, the kits provide at least one antibody-based binding moiety that binds to at least one biomarker protein, e.g. cortitropin releasing hormone (CRH), dehydroepiandrosterone (DHEA), neuropeptide Y (NPY) or Galanin, and a suitable container means. It is contemplated that in particular embodiments, the kit may further comprise a second antibody preparation (preferably detectably labeled) that binds immunologically to the same biomarker protein as the first antibody preparation, but where the first and the second antibodies bind to different epitopes; and a suitable container means thereof. In a particularly preferred aspects the first antibody preparation is attached to a support. It is contemplated that the support may be any support routinely used in immunological techniques. In a particularly preferred embodiments, the support independently is a polystyrene plate, test tube or dipstick. One preferred embodiment includes the use of a dip-stick.

The kits may include multiple antibodies that interact with each of the biomarker proteins of the invention, such that multiple biomarker proteins can be measured.

In other embodiments, the assay kits to measure the level of biomarker protein may employ (but are not limited to) the following techniques: competitive and non-competitive assays, radioimmunoassay (RIA) , bioluminescence and chemiluminescence assays, fluorometric assays, sandwich assays, immunoradiometric assays, dot blots, enzyme linked assays including ELISA, microtiter plates, and immunocytochemistry. For each kit the range, sensitivity, precision, reliability, specificity and reproducibility of the assay are established by means well known to those skilled in the art.

The above described assay kits would further provide instructions for use.

All references cited above or below are herein incorporated by reference.

The present invention is further illustrated by the following Examples. These Examples are provided to aid in the understanding of the invention and are not construed as a limitation thereof.

### EXAMPLE 1

### Biomarkers for Chronic Pelvic Pain Syndrome and Allostatic Overload

### Methods:

We evaluated the pre-(VB2) and post-prostatic massage (VB3) urine levels of several novel biomarkers of dysfunctional HPA axis activity. VB2 and VB3 samples were collected from 24 men with a diagnosis of end stage CP/CPPS (median age, 48 years) and 30 age-matched asymptomatic controls. Patients and controls underwent standardized clinical evaluation and were required to complete the National Institutes Chronic Prostatitis Symptom Index (NIH-CPSI).

The following biomarkers were measured in VB2 and VB3 using ELISA kits: corticotropin releasing hormone (CRH), neuropeptide Y (NPY), both obtained from Phoenix Pharmaceuticals, Belmont, Calif.), dehydroepiandrosterone (DHEA) and epidermal growth factor (EGF) from R & D Systems, Minneapolis, Minn. Galanin levels were measured using RIA (Phoenix Pharmaceuticals, Belmont, Calif.).

Modified allostatic load score was calculated based on a review of the neuroscientific literature.

### Results:

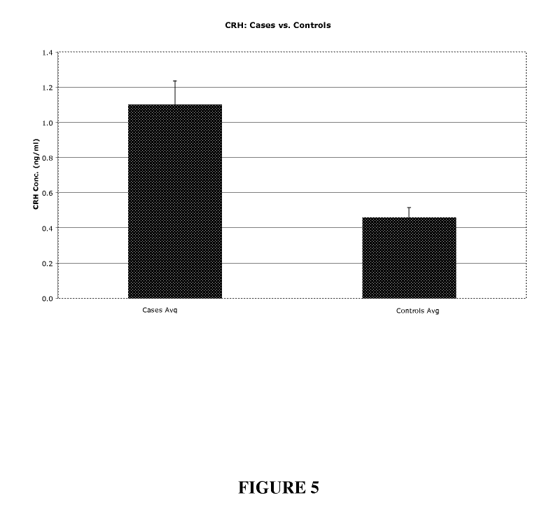

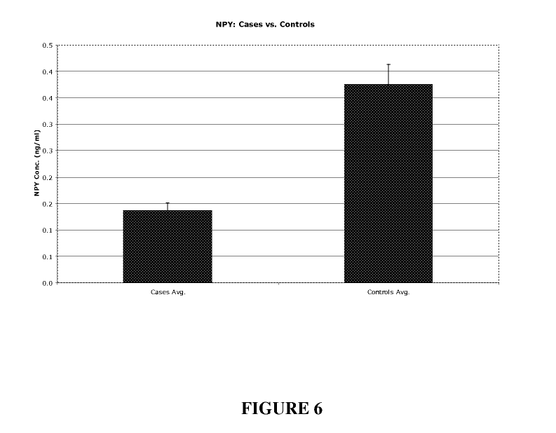

Allostatic load scores were significantly higher in patients with CPPS as compared with controls (P<0.001). Furthermore, PPMU NPY and galanin levels in CPPS patients were significantly lower as compared to those in controls. The PPMU of CRH and DHEA were significantly higher as compared with controls (P<0.001). See FIGS. 5 and 6 for an example of CRH and NPY levels respectively.

Chronic prostatitis/chronic pelvic pain syndrome (CP/CPPS) is a frustrating clinical condition manifested by pain or discomfort in the pelvic region for at least 3 months in the previous 6 months. Several lines of evidence suggest that abnormal neuroendocrine function may underlie CP/CPPS and CP/CPPS might be considered a “high sympathetic outflow” condition that is inadequately restrained by adrenocortical steroids due to a dysfunctional hypothalamic-pituitary-adrenocortical (HPA) axis. Our results show evidence of allostatic overload in CPPS patients as compared with controls and provide exciting insights into possible therapeutic avenues for the management of CPPS.

The references cited throughout the specification are hereby incorporated by reference.

We claim:

1. A method for facilitating the diagnosis of a subject for chronic prostatitis/chronic pain pelvic syndrome (CP/CPPS) in men or intersitial cyctitis (IC) in women comprising measuring the level of at least one biomarker protein in a urine test sample, wherein the biomarker protein is selected form the group consisting of cortitropin releasing hormone (CRH), dehydroepiandrosterone (DHEA), neuropeptide Y (NPY) and Galanin;
wherein, a higher level of cortitropin releasing hormone (CRH) or dehydroepiandrosterone (DHEA) in the test sample as compared to a reference level is indicative of CP/CPPS in men or IC in women, and wherein a lower level of neuropeptide Y (NPY) or Galanin in the test sample as compared to a reference level is indicative of CP/CPPS in men or IC in women.

2. The method of claim 1, wherein at least 2 biomarker proteins are measured.

3. The method of claim 1, wherein at least 3 biomarker proteins are measured.

4. The method of claim 1, wherein at least 4 biomarker proteins are measured.

5. The method of claim 1, wherein the presence of the biomarker protein is detected using an antibody-based binding moiety which specifically binds to the biomarker protein.

6. The method of claim 1, wherein the level of the biomarker protein is measured by measuring the activity of the biomarker.

7. The method of claim 5, wherein the level of biomarker protein is measured by a method comprising the steps of:
a. contacting the test sample, or preparation thereof, with an antibody-based binding moiety which specifically binds the biomarker protein to form an antibody-biomarker protein complex; and
b. detecting the presence of the complex, thereby measuring the level of biomarker protein present.

8. The method according to claim 5 or 7, wherein the antibody-based binding moiety is labeled with a detectable label.

9. The method according to claim 8, wherein the label is selected from the group consisting of a radioactive label, a hapten label, a fluorescent label, and an enzymatic label.

10. The method according to claim 5 or 7, wherein the antibody-based binding moiety is a monoclonal antibody.

11. A kit designed for facilitating the diagnosis of chronic prostatitis/chronic pain pelvic syndrome (CP/CPPS) in men or intersitial cyctitis (IC) in women comprising a means for detecting, in urine, one or more protein biomarkers selected from the group consisting of cortitropin releasing hormone (CRH), dehydroepiandrosterone (DHEA), neuropeptide Y (NPY) and Galanin; a container for holding a urine sample; and instructions for use, wherein the means for detecting one or more of the protein biomarkers comprises an antibody-based binding moiety that specifically binds to the biomarker/s to be detected.

12. The kit of claim 11, comprising at least two, at least three, or at least 4, antibody-based binding moieties which detect the presence or levels of at least two, three, or four protein biomarkers.

13. The kit of claim 11, wherein the kit comprises at least two antibodies, one antibody is immobilized on a solid phase and one antibody is detectably labeled.


SEQUENCE LISTING (4 sequences)

SEQ ID NO: 1 - protein, 196 aa, Homo sapiens
MRLPLLVSAGVLLVALLPCPPCRALLSRGPVPGARQAPQHPQPLDFFQPPPQSEQPQQPQARPVLLRMGEEYFLRLGNLNKSPAAPLSPASSLLAGGSGSRPSPEQATANFFRVLLQQLLLPRRSLDSPAALAERGARNALGGHQEAPERERRSEEPPISLDLTFHLLREVLEMARAEQLAQQAHSNRKLMEIIGK

SEQ ID NO: 2 - protein, 285 aa, Homo sapiens
MSDDFLWFEGIAFPTMGFRSETLRKVRDEFVIRDEDVIILTYPKSGTNWLAEILCLMHSKGDAKWIQSVPIWERSPWVESEIGYTALSETESPRLFSSHLPIQLFPKSFFSSKAKVIYLMRNPRDVLVSGYFFWKNMKFIKKPKSWEEYFEWFCQGTVLYGSWFDHIHGWMPMREEKNFLLLSYEELKQDTGRTIEKICQFLGKTLEPEELNLILKNSSFQSMKENKMSNYSLLSVDYVVDKAQLLRKGVSGDWKNHFTVAQAEDFDKLFQEKMADLPRELFPWE

SEQ ID NO: 3 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 4 - protein, 123 aa, Homo sapiens
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS